(19) United States
(12) Patent Application Publication
(10) Pub. No.: US 2015/0164372 A1
(43) Pub. Date: Jun. 18, 2015

(54) INTELLIGENT IMPLANTED HEALTH SENSING DEVICE AND ASSEMBLY

(71) Applicants: Nassir NAVAB, Munich (DE); Amin KATOUZIAN, Munich (DE)

(72) Inventors: Nassir Navab, Munich (DE); Amin Katouzian, Astoria, NY (US); Reza Ghotbi, Munich (DE); Christoph Hennersperger, Munich (DE); Michael Friebe, Munich (DE)

(73) Assignees: Amin KATOUZIAN, Astoria, NY (US); Nassir NAVAB, Munich (DE)

(21) Appl. No.: 14/415,232

(22) PCT Filed: Jul. 19, 2013
(86) PCT No.: PCT/EP2013/065322

§ 371 Date: Jan. 16, 2015

(30) Foreign Application Priority Data
Jul. 20, 2012 (EP) 12177301.4

(51) Int. Cl. A61B 5/07; G01R 33/48; A61F 2/82; A61B 5/00; A61F 2/06; A61B 5/0215; G01R 33/28

(52) U.S. Cl. CPC A61B 5/076; A61B 5/02158; G01R 33/48; G01R 33/287; A61B 5/4851; A61B 5/6898; A61B 5/686; A61F 2/06; A61F 2/82

(57) ABSTRACT

A medical device configured for monitoring one or more health conditions, particularly restenosis, as described. The medical device includes two or more sensors configured to be implanted for exposure to blood within a vessel or to blood within a graft provided in a vessel, wherein each of the two or more sensors is configured to sense at least the pressure in the vessel or the graft; and a support structure for supporting the two or more sensors, wherein the support structure has a distal end and a proximal end, wherein at least one of the two or more sensors is closer to the distal end than another one of the two or more sensors.

### TECHNICAL FIELD OF THE INVENTION

Embodiments of the present invention relate to devices and methods for monitoring vascular health conditions, e.g. restenosis and/or thrombosis. Embodiments of the present invention particularly relate to sensor arrangements in support structures configure for implantation, e.g. stents, and/or to communication techniques of sensor arrangements utilized in such support structures. Specifically, they relate to medical devices configured for monitoring one or more health conditions, particularly restenosis, and to methods of monitoring a health condition, particularly restenosis.

### BACKGROUND OF THE INVENTION

The word “atherosclerosis” comes from the Greek words “athero” and “sclerosis”, meaning gruel or paste and hardness, respectively. As it is inferred from the name, it is a disease in which atheromatous plaque, consisting of fatty substances (i.e. cholesterol), platelets, cellular waste products, and calcium build up in the innermost layer of an artery called endothelium. The disease is typically asymptomatic, chronic, slowly progressive, cumulative, and eventually leads to plaque ruptures and therefore clots inside the artery lumen cover the ruptures. The clots heal and usually shrink but leave behind stenosis (narrowing) or in worst case scenario complete occlusion of the artery.

Atherosclerotic disease may occur anywhere throughout vascular system. For example, coronary heart disease (CHD) that is the most common type of heart disease takes about 500,000 lives in the United States every year. In practice, coronary atherosclerotic disease (CAD) is treated by interventional radiologists or cardiologists through medication, non-invasive, or invasive procedures. The choice of physical treatment approach depends on severity of extended disease, which could lead to minimally invasive (e.g. angioplasty, stent implantation), or invasive (e.g. bypass, heart transplantation) surgical procedures.

Atherosclerosis could occur in peripheral arteries that supply blood from heart to head, arms, legs, kidneys and stomach. Similar to CAD, a minimal invasive procedure could be performed to open up the blockage through angioplasty or stent implantation. Another disease that is also correlated with atherosclerosis is abdominal aortic aneurysm (AAA) that is enlargement of abdominal aorta for more than 50% of its original diameter. AAA is the 13th leading cause of death in the US and about 15000 Americans die each year. It is also a chronic disease and when it ruptures only 10-25% of patients survive leaving 75-90% fatality. Likewise CAD and peripheral atherosclerotic disease (PAD), the AAA is stabilized through stent implantation procedure too, known as endovascular aortic (aneurysm) repair (EVAR).

For many years, the ultimate goal of an interventional cardiologist was to physically expand narrowed artery, using balloon and stent, or create additional blood supply connections through bypass surgery. With refinement of new intravascular imaging modalities, today, primary responsibility of an interventional cardiologist is not only to open up the site of occlusion but also to properly cure the atherosclerosis. For example, the United States food and drug administration (FDA) safety panel has suggested that cardiologists take more measures to reduce risks associated with the stents due to troubling headlines about potentially deadly clotting risks in small percentage of them. So far, researchers have mainly focused on the stents themselves and how they are made. But now, attention is turning more toward the way they are being used. There has been a consensus that widespread use of drug—coated stents may increase the risk of deadly clots formation, which has called attentions in the United States and widely reported in the media (Fox News report, Oct. 9, 2008—The New York Times, Feb. 13, 2007—The Wall Street Journal, May 7, 2007—The Boston Globe, Dec. 26, 2004) and in many scientific journals. The medicine on the stent is released gradually over time to stop the progression of the scar tissue. However, the artery can become narrow again, which is known as restenosis. Currently, there is no systematic approach to monitor the progression of atherosclerosis or any abnormality like thrombosis within implanted stent and early detection of restenosis or possible myocardial infarction.

In view of the above, it is an object of the present invention to provide a devices and methods for monitoring restenosis and/or thrombosis and related disease that overcome at least some of the problems in the art.

### SUMMARY OF THE INVENTION

In light of the above, a device according to independent claims 1 and 3, and a method according to independent claim 11 are provided. Further aspects, advantages, and features of the present invention are apparent from the dependent claims, the description, and the accompanying drawings.

According to one embodiment a device or assembly configured for monitoring one or more health conditions, particularly restenosis, is provided. The device or assembly includes: two or more sensors configured to be implanted for exposure to blood within a vessel or to blood within a graft provided, e.g. implanted, in a vessel, wherein each of the two or more sensors is configured to sense at least the pressure in the vessel or the graft, a support structure for supporting the two or more sensors, wherein the support structure has a distal end and a proximal end, wherein at least one of the two or more sensors is closer to the distal end than another one of the two or more sensors.

According to another embodiment, a device or assembly configured for monitoring one or more health conditions, particularly restenosis, is provided. The device includes one or more sensors configured to be implanted in a vascular, wherein the one or more sensors are configured to sense at least pressure values, a support structure for supporting the one or more sensors, and an a remote device for wirelessly providing energy to the one or more sensors and for reading the pressure values.

According to another embodiment, a device or assembly configured for monitoring one or more health conditions, particularly restenosis, is provided. The device includes one or more electronic chips integrated with sensors configured to be implanted in a vascular, wherein the one or more electronic chips are configured to transmit one or more sensed indices wirelessly, e.g. at least pressure values, a support structure for supporting the one or more sensors and chips, and an a remote device for wirelessly providing energy to the one or more sensors and for reading the pressure values.

According to a further embodiment, a method of monitoring a health condition, particularly restenosis, is provided. The method includes: wirelessly energizing at least one sensor, which senses at least vascular pressure values, with a remote device, reading at least the pressure values for a predetermined time period via a wireless data transfer from the at least one sensor, communicating the pressure values to a computer system, and receiving information related to the health condition from the computer system.

According to one embodiment a device or assembly configured for monitoring one or more health conditions, particularly restenosis, is provided. The device or assembly includes: two or more sensors configured to be implanted for exposure to blood within a vessel or to blood within a graft provided, e.g. implanted, in a vessel, wherein each of the two or more sensors is configured to sense at least the pressure in the vessel or the graft, a support structure having two or more rings configured to be implanted in a vessel and for supporting the two or more sensors, wherein the support structure has a distal end and a proximal end, wherein at least one of the two or more sensors is arranged at a first ring of the two or more rings, which is closer to the distal end than a second ring of the two or more rings, wherein at least another sensor of the two or more sensors is attached to the second ring, and a remote device for wirelessly providing energy to the one or more sensors and for reading the pressure values and a reading device configured for wireless communication with the two or more sensors and comprising a port for wired or wireless communication with a computer system, wherein the computer system is a system selected from the group consisting of: a personal computer, a portable computer, a smartphone, a server, a cloud computer system, and combinations thereof.

Embodiments are also directed at apparatuses for carrying out the disclosed methods and include apparatus parts for performing each described method step. These method steps may be performed by way of hardware components, a computer programmed by appropriate software, by any combination of the two or in any other manner. Furthermore, embodiments according to the invention are also directed at methods by which the described apparatus operates. It includes method steps for carrying out every function of the apparatus.

### BRIEF DESCRIPTION OF THE DRAWINGS

So that the manner in which the above recited features of the present invention can be understood in detail, a more particular description of the invention, briefly summarized above, may be had by reference to embodiments. The accompanying drawings relate to embodiments of the invention and are described in the following:

FIGS. 1A to 1D show medical devices including stents as support structures and sensors according to embodiments described herein;

FIG. 2 shows a medical device including a stent assembly as a support structure and sensors according to embodiments described herein;

FIGS. 3A to 3C illustrate the monitoring of restenosis with medical devices according to embodiments described herein and with methods according to embodiments described herein;

FIG. 4 illustrates a system including a stent, a sensor, a remote device for reading sensor values, and a computer system for monitoring a health condition, e.g. restenosis, according to embodiments described herein;

FIGS. 5 and 6 illustrate yet further systems including support structures, at least one sensor, a remote device for reading sensor values, and a computer system for monitoring a health condition, e.g. restenosis, according to embodiments described herein;

FIGS. 7A and 7B illustrate rings that can be used to form support structures for embodiments described herein;

FIGS. 8A to 8F illustrate sensors mounted to a ring for use in embodiments described herein;

FIG. 9 shows a medical device including a support structure and sensors according to embodiments described herein;

FIGS. 10A to 10C illustrate the monitoring of restenosis with medical devices according to embodiments described herein and with methods according to embodiments described herein;

FIG. 11 illustrate a flow chart for explain the benefits for treatment of e.g. restenosis and further deployment of angioplasty with medical devices according to embodiments described herein and with methods according to embodiments described herein;

FIGS. 12A and 12B illustrate systems including a support structure, sensors, a remote device for reading sensor values, and a computer system for monitoring a health condition, e.g. restenosis, according to embodiments described herein; and

FIGS. 13A and 13B illustrate yet further systems including support structures, at least one sensor, a remote device for reading sensor values, and a computer system for monitoring a health condition, e.g. restenosis, according to embodiments described herein.

### DETAILED DESCRIPTION OF EMBODIMENTS

Reference will now be made in detail to the various embodiments of the invention, one or more examples of which are illustrated in the figures. Within the following description of the drawings, the same reference numbers refer to same components. Generally, only the differences with respect to individual embodiments are described. Each example is provided by way of explanation of the invention and is not meant as a limitation of the invention. Further, features illustrated or described as part of one embodiment can be used on or in conjunction with other embodiments to yield yet a further embodiment. It is intended that the description includes such modifications and variations.

According to embodiments described herein, an intelligent implantable health sensing system is provided. The health sensing system can thereby, include one chip such as a RFID chip, or typically two or multiple RFID chips within support structure's length, e.g. stent's length, to measure and record spatially relative pressure differences. The one or more RFID chips may be integrated with a sensor. Measuring pressure differences helps to monitor any abnormality (restenosis, hemorrhage) systematically for early detection of restenosis and/or failure of a stent. Further, development of disease- and patient-specific cardiac models for early detection of myocardial infarction can be based upon embodiments described herein.

According to yet further embodiments, which can be combined with other embodiments described herein, at least one of the two or more sensors is mounted to the support structure adjacent to the distal end, and wherein the another one of the two or more sensors is mounted to the support structure adjacent to the proximal end. The sensors can be employed as radiofrequency coils in magnetic resonance (MR) imaging systems. The sensors and/or chips, which may be further integrated with further structures can be used as MRI coupling coils (passive coupling through a LC circuit tuned to the MRI lamor frequency) for intravascular imaging. This can provide imaging information about a stenosis grade and also about the structure of the plaque.

The health sensing system is supported with a user-interaction device, e.g. a remote device. According to typical embodiments, which can be combined with other embodiments described herein, the user-interaction device can be a computer or a mobile device, e.g. a smartphone and can optionally, be provided with back-end architecture for secure communication and recording of data and online/offline disease-, patient-, or patient-flow-modeling.

Embodiments described herein can provide a device in which senses the progression of restenosis and/or hemorrhage within the implanted stent upon user's request and sends this information to an external near-body device (e.g. a smartphone). The data can, for example, then transferred to a centralized database for online processing and decision-making. According to some embodiments, which can be combined with other embodiments described herein, two or multiple chips (sensors) are designed along with a stent to collect pressure indices and/or temperature indices, flow rate indices, and/or possible biomarkers.

According to some embodiments, two (or multiple) pressure sensors are designed on stent or another support structure and the pressure indices will be fetched through an external near-body device, such as a remote device including a reading device. The data is transferred to a centralized data station for online analysis and eventually a report is created. In case of life threatening situation, an alarm can be sent to an expert.

According to typical embodiments, at least two adjacent to at proximal and distal points of a stent are provided, which are configured to monitor the relative pressures difference between the two sensors. Multiple chips can also be designed within a stent another or support structure and therefore spatially relative pressures will be measured and recorded for any abnormality identification.

According to some embodiments, which can be combined with other embodiments described herein, the devices and systems employ multiple sensors and measure the spatially relative pressure differences. Accordingly, variations in blood pressure due to stress or other abnormal health conditions can be compensated as the pressure difference at two predetermined positions, e.g. a proximal and distal end of a stent or a graft, is measured. Hence, embodiments facilitate detection of any abnormality within the stent in a simpler and more reliable manner compared to measurements with one sensor. Furthermore, in case one of the sensing devices would be malfunctioning due to a failure or due to extensive plaque covers on one of the sensing devices (chips), the other(s) sensing devices can continue providing information.

According to some embodiments, which can be combined with other embodiments described herein, the devices and systems employ multiple sensors and/or chips and measure the spatially relative pressure differences. Accordingly, the first measurements right after implantation can be deploy for data calibration. The data calibration includes measured relative pressure differences, which are served to assess disease severity under different psychological and physical conditions (e.g. rest, exercise, stress, etc.). For example, saturation of sensors can be an indicative for abrupt or gradual changes in disease severity.

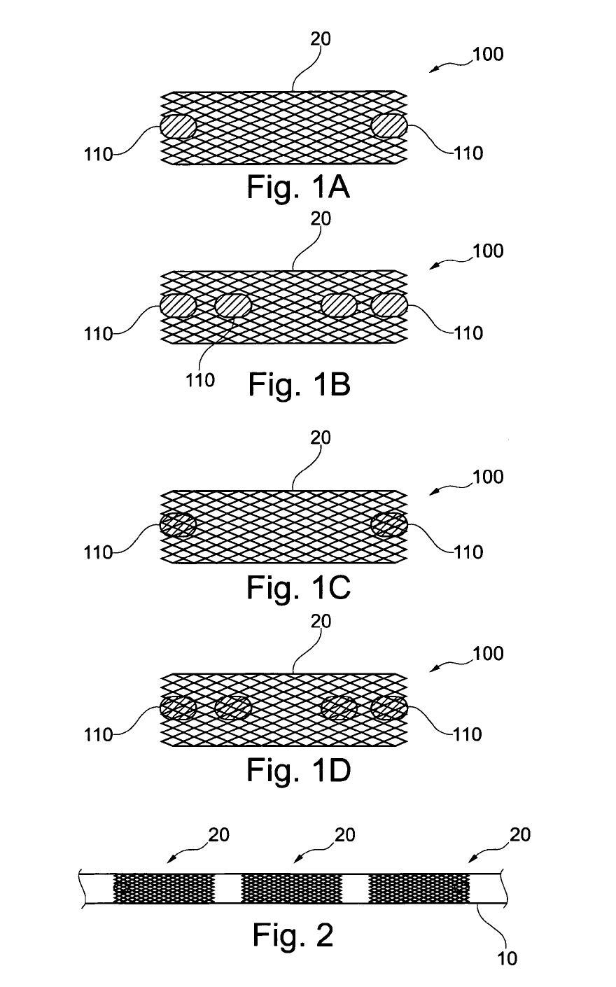

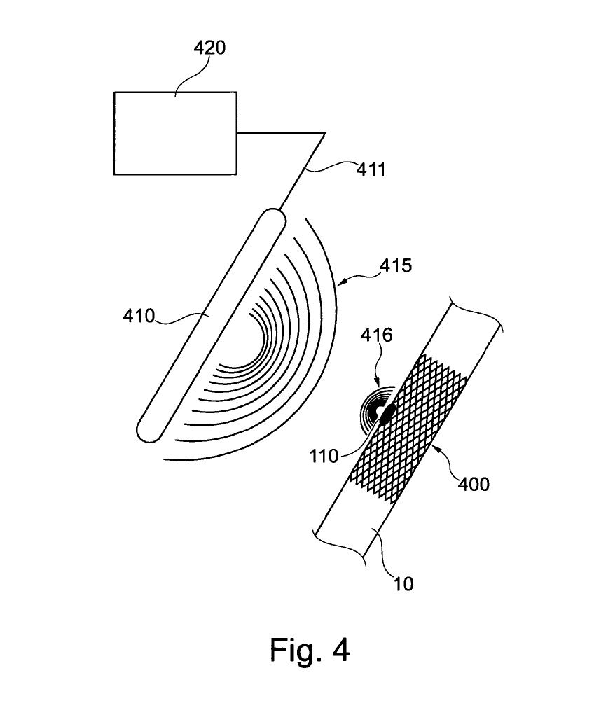

FIG. 1A illustrates a first embodiment of a medical device (100). FIG. 1A shows two sensors 110 mounted in vicinity of opposing ends of a support structure, e.g. a stent 20. The stent with the sensors attached thereto can be implanted in a blood vessel according to general usage of a stent for vascular disease. The sensors 110 are provided in longitudinal direction of a blood vessel and can, thus, be configured to provide a pressure value at a first longitudinal position and a second longitudinal position. According to embodiments described herein, the pressure difference can be sensed to monitor the health condition as described in more detail with respect to FIGS. 3A to 3C.

According to typical embodiments, which can be combined with other embodiments described herein, the sensors 110 can be radio-frequency chips or integrated with radio-frequency chips, similar to RFID chips. Thereby, the sensors are powered by an external RF source, e.g. in the GHz range, such as 2 GHz to 100 GHz. On receiving energy by the external source, the sensors 110 provide pressure values of the actual pressure at the sensor. The pressure values are transmitted with a wireless connection to an external reading device. According to a typical example, the external RF source and the reading device can be integrated in one remote device for powering and reading the sensors.

During operation such a remote device or the respective RF source and the reading device can be held next the patient's body part, in which the stent 20 with the sensor 110 is implanted for a predetermined period of time. The pressure values can e.g. be read during a time period ranging from 5 seconds to 5 minutes, e.g. 2 min. A few heart cycles can be measured in that time period. Thereby, statistically improved pressure values, e.g. average values or values optimized by other statistic models (e.g. variance correlation) and signal processing technique (e.g. spectral analysis) can be obtained. The limitation to a predetermined time period further has the benefit that possible heat generation due to energy consumption of the chip can be limited to a time span, which is harmless to the tissue of the blood vessel in which the stent is implanted.

As shown in FIG. 1A, two sensors can be provided adjacent to the ends of the support structure, e.g. stent 20. According to yet further embodiments, which can be combined with other embodiments described herein, more than two sensors 110 can be provided. As shown in FIG. 1B for example four sensors 110 can be provided. However, any other suitable number of sensors 110 can be provided, wherein the number may depend on the longitudinal length of the stent 20. For embodiments, including two sensors 110 or even one sensor 110, the data transmitted from the sensors to the reading device can consist of the pressure values as a function of time, yet, more information may be included as described in more detail below. The reduction to a pair (sensor 1 and sensor 2) of pressure values or a single pressure values (one sensor) is possible, as only one value is provided or only a pressure difference is considered. For embodiments including more than two sensors 110, typically a sensor ID is further transmitted by each of the sensors. Thereby, it is possible to calculate pressure difference between specific ones of the sensors such that the pressure differences, which can be calculated from the plurality of timely corresponding pressure values do not get confused and can be correlated to a specific pair of sensors 110.

As shown in FIGS. 1A and 1B, the sensors 110 are provided at the outside of the stent 20. Yet, as the stent 20 has a mesh forming the stent, the sensors are exposed to the blood in the vasculature. FIGS. 1C and 1D show corresponding embodiments of medical devices 100, where the sensors 110 are provide at the inside of the stent. This arrangement is, for example, used when a membrane is used with the stent or a graft having a membrane is used as a support structure. Thereby, the blood pressure within the vasculature, i.e. for example not in the sac of an aneurysm, is measured by the sensors 110 towards the inside side of the membrane.

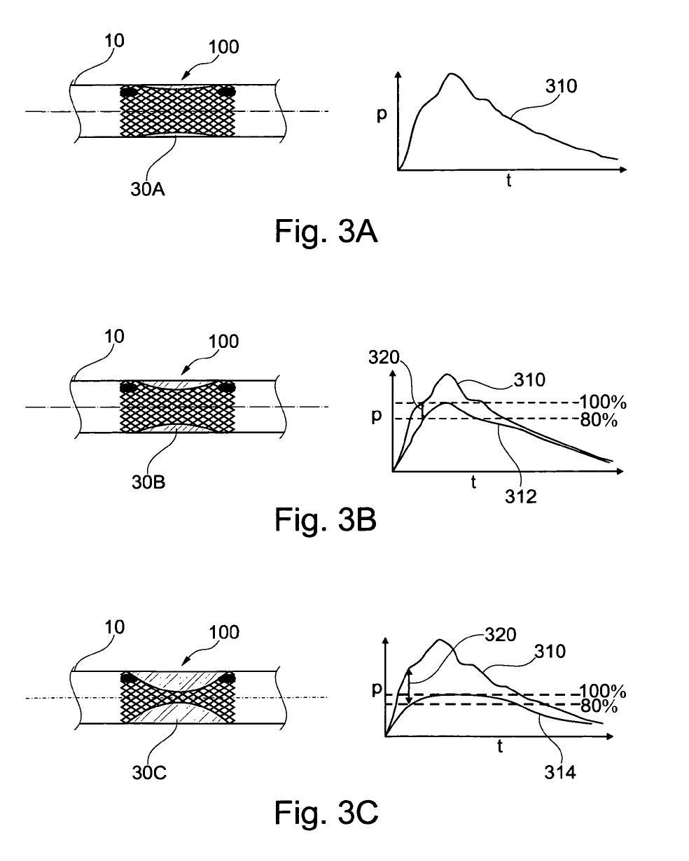

As described above, the support structure, which is configured to be implanted in a human or animal body together with the sensors 110, can be provided by a stent or a graft. However, according to yet further embodiments, which can be combined with other embodiments described herein, the support structure can also be provided by a stent arrangement comprising two or more stents. FIG. 2 shows an example of a medical device with a support structure including three stents 20. Thereby, three stents 20 are shown as implanted in a blood vessel along a longitudinal direction of the vessel 10. The medical device includes two sensors 110, which are arranged adjacent a distal and proximal end of the stent arrangement. However, according to yet further embodiments, the sensors can have any predetermined longitudinal position along the support structure, e.g. the stent assembly, which provides a sufficient distance between a first sensor and a second sensor to sense a pressure difference indicative of the health condition to be monitored. Thereby, according to typical embodiments described herein, which can be combined with other embodiments described herein, the predetermined position is defined merely by a longitudinal position, i.e. a rotation along the longitudinal axis is not considered and measurement results are irrespective thereof. This can, for example, be provided by the fact that pressure differences are considered.

FIGS. 3A to 3C show a medical device 100 provided in a blood vessel 10, wherein restenosis indicated by areas 30A, 30B and 30C increasingly occurs from 30A to 30B and further to 30C. Thereby, FIG. 3A shows a healthy condition or a condition which is almost healthy when considering a stent implantation. FIG. 3B shows a growing restenosis. FIG. 3C shows a critical restenosis. As shown by the curves 310, 312 and 314, the growing restenosis results in a reduced pressure value at the respective sensor 110 of the medical device 100. As indicated by arrow 320, a pressure difference between the sensor values corresponding to a time t is determined and the health condition, e.g. the extent to which restenosis occurs is determined by the pressure difference indicated by arrow 320. According to different embodiments, which can be combined with other embodiments described herein, the pressure difference can be considered at 60% to 90%, e.g. 80% of the peak value (100%), either of the lower pressure peak as shown in the figures or of the higher pressure peak. The pressure functions shown in the graphs of FIGS. 3A to 3C basically correspond to the pressure values during one heartbeat. A measurement with a remote reading device is typically conducted for a longer time interval, e.g. 10 sec to 3 min, such that a more accurate measurement can be conducted as described above. Accordingly to typical examples, which can yield further embodiments, with the embodiments disclosed herein, the data rate transmitted from a sensor to the remote device can range from 20 to 100 Kbit per second, for example 40-50 Kbit/s. According to some embodiments, which can be combined with other embodiments described herein, the restenosis can be detected within the support structure, e.g. a stent and/or between the at least two sensors. Alternatively, it is possible to use the plurality of pressure values also to sense restenosis or similar abnormality before or after the stent in the vicinity thereof.

FIG. 4 illustrates yet further embodiments described herein. A medical device for implantation 400 includes a support structure, e.g. a stent, a stent arrangement, a graft, or the like, and at least on sensor 110 mounted to the support structure. The sensors according to embodiments described herein typically have not internal power supply. The sensor 110 is configured to sense at least the pressure inside a vascular. Optionally, further biological indices of interest can be included like one or more of a temperature, a flow rate or detection of biomarkers can be conducted as well. Thereby, for those embodiments having two or more corresponding sensors, value differences are considered at least for the pressure, the temperature and/or the flow rate.

According to typical embodiments described herein, the sensor can be provided by a MEMS (micro-electro-mechanical system). Thereby, a chip can be provided with a pressure sensor, a wireless power supply (see, e.g. RFID), a microelectronics and an antenna for transmitting the pressure values and/or further parameter values, or even a sensor ID to an external receiver or reading device. Accordingly, the sensor can include an HF frontend module for transmitting the sensed values as a function of time. The entire electronic can be provided on the chip. The sensor or chip further includes the measurement device or mechanical device, e.g. the pressure gauge, a wireless power supply, wherein the energy is transmitted by RF radiation, e.g. in the GHz range to the sensor, and optionally also a control unit and/or evaluation unit. According to alternatives a control unit and/or evaluation unit can also be provided in a remote reading device receiving the data.

As shown in FIG. 4, RF radiation 415 is emitted by a remote device 410. The RF radiation is used to energize the sensor 110, which in turn transmits data of measurement values and optionally other information as a wireless signal to the remote device 410 as indicated by signal 416. The remote device 410 includes the transmitter for the RF energy as well as a reading device for reading the data transmitted from the sensor 110. Alternatively, the remote RF transmitted and the reading device can also be separated. The remote device 410 is connected to a computer system 420 via connection 411. The computer system can be a personal computer, a portable computer, a server structure, a cloud structure or combinations thereof. Typically, utilizing a back-end portal structure with encrypted communication techniques allows for further data processing. Thereby, historical information and current information can be shared and compared and additional information like medication or the like can be considered in a model calculation. Those results can be shared with the user of the remote device 410 and/or a medical scientist (doctor). If critical or even emergency conditions are found, the patient, typically the user of the remote device, and/or the doctor can be informed or an alert could be generated.

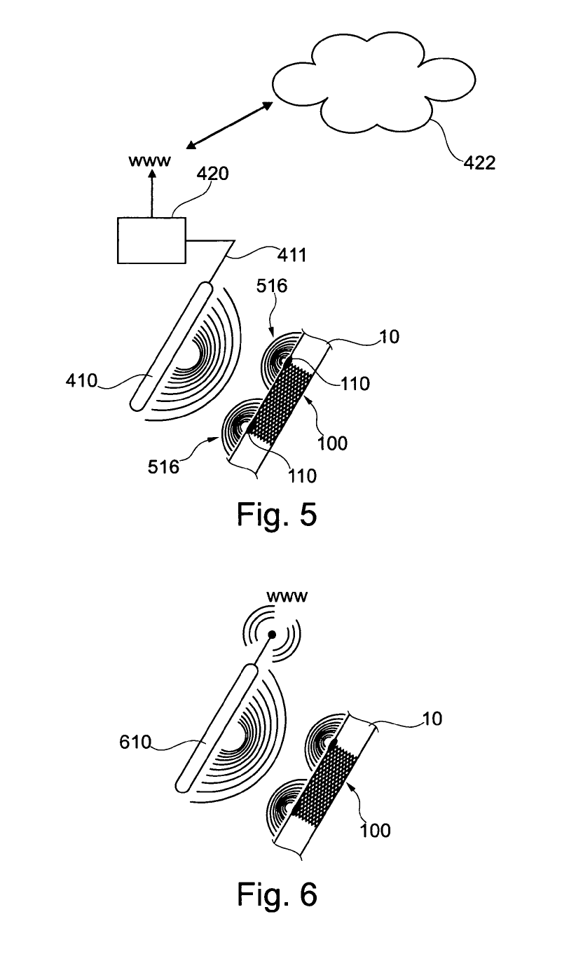

FIG. 5 shows an embodiment where a medical device 100 having two sensors 110 is provided in a blood vessel 10. The details for power supply and data transfer from the remote device 410 to the sensors 110, which have been described above with respect to FIG. 4 can also be incorporated in the embodiments described with respect to FIG. 5 to yield yet further embodiments. The remote device 410 is connected to a computer system 420, e.g. a laptop, which is connected to the World Wide Web and server structure, e.g. in the form of a cloud solution 422. As indicated by the arrows in FIG. 5, the server structure can communicate back to the user. The cloud solution facilitates sharing of information, e.g. with a medical expert.

FIG. 6 illustrates yet a further embodiment, where the remote device having the RF transmitter for energy transfer and the reading device for data receipt incorporated in a mobile device, such as a smart phone or the like, which can directly communicate with servers provided in a network. Modifications and implementations described above with respect to sensor arrangement, measurement methods and sensor chip design and wireless communication techniques can likewise be applied for solutions shown in FIGS. 5 and 6.

As described above, a support structure, which is configured to be implanted in a human or animal body together with the sensors 110, is provided. Thereby, as already shown in FIG. 2, the support structure can have two or more elements. According to some typical implementations, the sensors can be provided on different ones of the two or more elements.

According to some embodiments, which can be combined with other embodiments described herein, the support structure can include: a stent, a stent arrangement, a graft, a graft arrangement, two or more rings, or two or more plates, e.g. plate patches, or other elements or medical devices that can be inserted in a vessel. Thereby, according to some additional or alternative implementations, a ring can be provided as a short stent, i.e. a stent with a length in axial direction of 5 mm or below. Unlike stents, the rings are not employed or configured to push away plaques toward arterial walls and open up the site of occlusion but employed through balloon angioplasty by placing them at distal or proximal ends of an inflating balloon. The primary role of the rings is to facilitate online monitoring of disease progression through presented embodiments. The rings and support structures can according to some embodiments, which can be combined with other embodiments described herein, be self-expandable. According to typical embodiments, which can be combined with other embodiments described herein, the ring can be round, oval or any other shape configured to be arranged in a vessel of an animal or human.

According to yet further embodiments, the sensors can have any predetermined longitudinal position along the support structure, e.g. the stent arrangement or ring arrangement, which provides a sufficient distance between a first sensor and a second sensor to sense a pressure difference indicative of the health condition to be monitored. Thereby, according to typical embodiments described herein, which can be combined with other embodiments described herein, the predetermined position is defined merely by a longitudinal position, i.e. a rotation along the longitudinal axis is not considered and measurement results are irrespective thereof. This can, for example, be provided by the fact that pressure differences are considered.

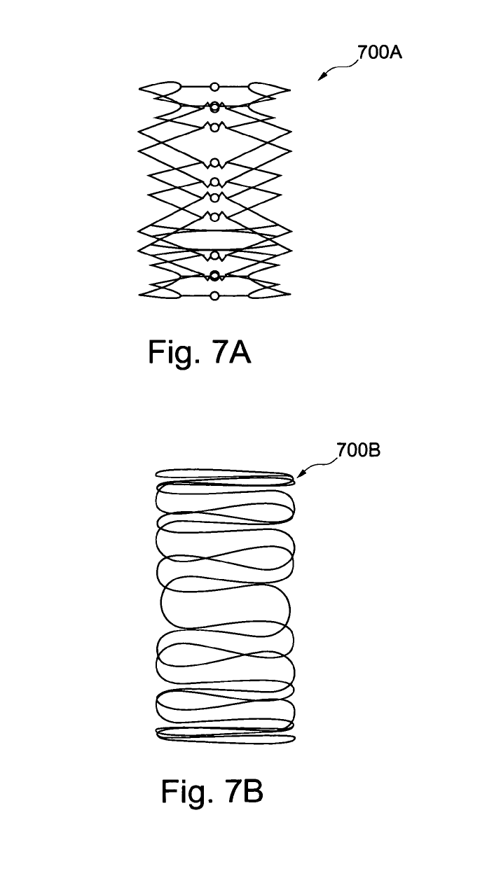

FIGS. 7A and 7B show pictures of rings 700A and 700B respectively. As can be seen, the rings 700A and 700B have a similar structure as a stent. However they are comparably short in axial direction. According to one definition, a ring could have a dimension in axial direction, which is shorted than the diameter of the ring.

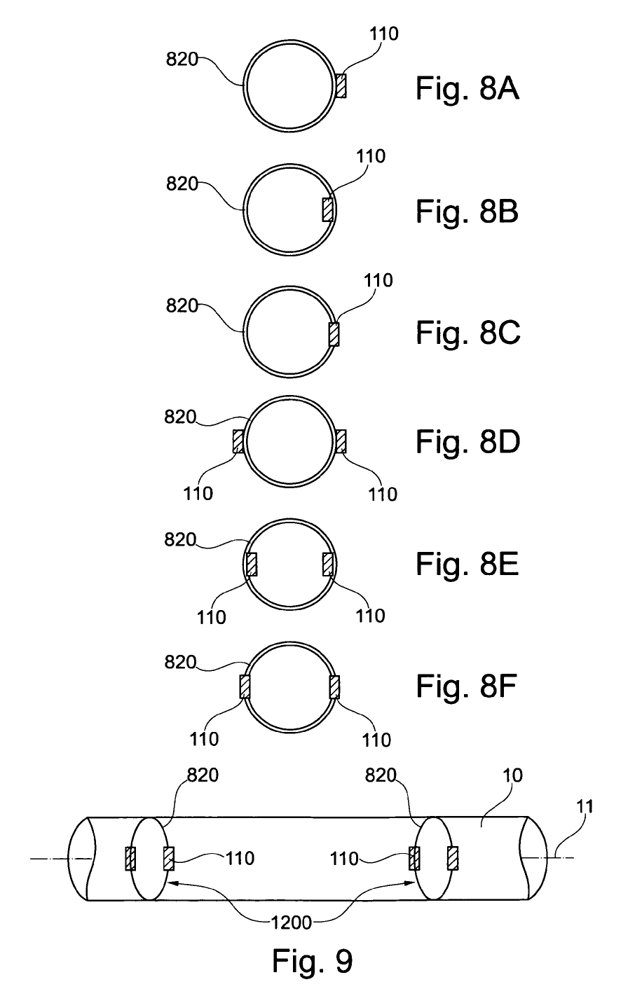

FIGS. 8A to 8F show different variations of providing sensors 110 provided at a ring 820. FIGS. 8A to 8C show embodiments, for which one sensor 110 is attached or mounted to the ring 820. Thereby, according to different embodiments, the sensor can be provided to be essentially radially outward of the ring 820 (see FIG. (A), essentially radially inward of the ring 820 (see FIG. 8C), or on the ring or within the ring. For example, the ring can be closed by the sensor, i.e. the ring is attached on two sides of the sensors (see FIG. 8B). For embodiments as illustrated by FIG. 8B, the sensor has essentially the same radial position as the ring.

According to yet further embodiments, as illustrated in FIGS. 8D to 8F, two sensors 110 can be provided on one ring 820. Similar to the description with respect to FIGS. 8A to 8C, the sensors 110 can be provide at different radial positions with respect to the ring. That is, the sensors 110 can be provided essentially radially outward (see FIG. 8D), essentially radially inward (see FIG. 8F, or essentially at a similar radial position as the ring (see FIG. 8E). It is to be understood that for two or more sensors, also different positions with respect to the ring 820 can be provided for one ring, i.e. one sensor is essentially radially inward and one sensor is essentially radially outward, etc.

Further, according to yet further embodiments, also 3, 4 or 5 sensors can be provided at one ring. Providing two or more sensors at the ring can increase the measurement accuracy because more than one signal can be obtained at one axial position, i.e. the axial position of the ring having the two or more sensors. Further, redundancy is provided in the event one of the two or more sensors fails to provide reliable signals, e.g. because of a failure or by coverage of plaque. In case one of the sensing devices would be malfunctioning due to a failure or due to extensive plaque covers on one of the sensing devices (chips), the other(s) sensing devices can continue providing information.

According to some embodiments, which can be combined with other embodiments described herein, two rings 820, each with at least one sensor 110, forms a medical device 1200, as e.g. shown in FIG. 9. Thereby, the support structure of the medical device can be provided by two or more rings. The two rings 820, wherein each ring 820 has two sensors 110 or sensing devices mounted thereon are configured to be implanted in a blood vessel along a longitudinal direction or axis 110 of the vessel 10. It is to be understood that even though the axis 110 is shown as a straight line in FIG. 9, the axis 110 is defined as following a potential curvature of the vessel 10. Thereby, the two rings 820 each having at least one sensor 110 form the medical device 1200 according to embodiments described herein. The rings can be provide at predetermined positions along the axis 11 or at a predetermined distance along the axis 11 in order to be able to monitor the health condition with respect to plaque coverage of vessel 10.

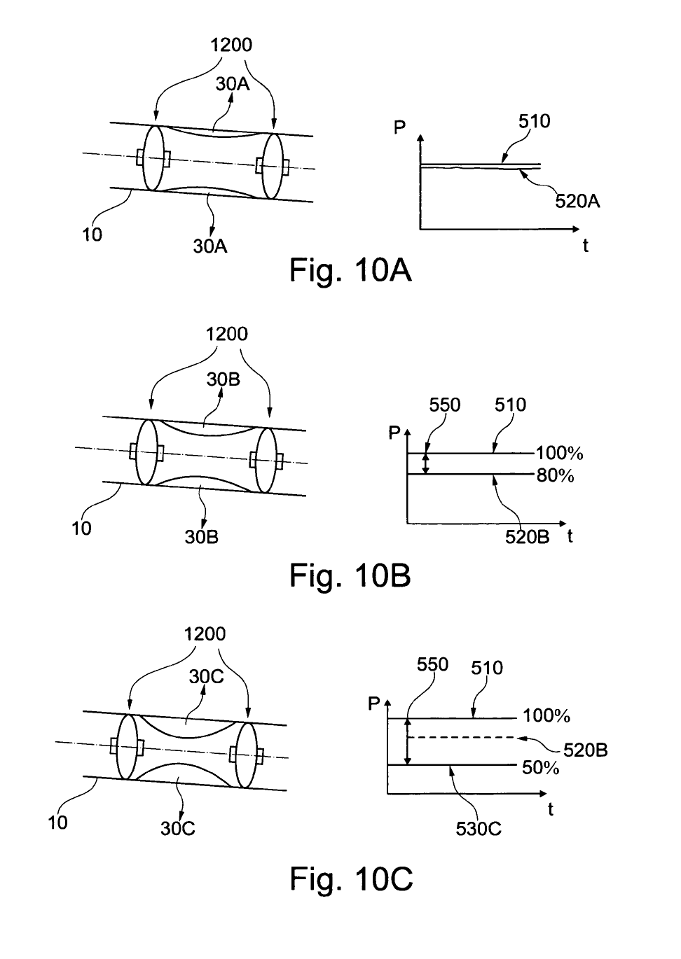

FIGS. 10A to 10C show a medical device 1200 provided in a blood vessel 10, wherein stenosis or restenosis indicated by areas 30A, 30B and 30C increasingly occurs from 30A to 30B and further to 30C. Thereby, FIG. 10A shows a healthy condition or a condition which is almost healthy when considering a surgery. FIG. 10B shows a growing restenosis. FIG. 10C shows a critical restenosis. As shown by the pressure levels 510, 520A, 520B and 520C, the growing restenosis results in a reduced pressure value at the respective sensor of the medical device 1200. As indicated by arrow 550, a pressure difference between the sensor values is determined and the health condition, e.g. the extent to which restenosis occurs is determined by the pressure difference indicated by arrow 550. According to different embodiments, which can be combined with other embodiments described herein, the pressure difference can be considered to have a threshold at 50% to 90%, e.g. 80% of the higher pressure. Accordingly, if the pressure difference exceeds a certain value or if the lower value 520A,B,C drops below a certain percentage of the higher value 510, a critical conditions is detected. According to some embodiments, which can be combined with other embodiments described herein, the restenosis can be detected within the support structure, e.g. a stent and/or between the at least two sensors. Alternatively, it is possible to use the plurality of pressure values also to sense restenosis or similar abnormality before or after the stent in the vicinity thereof.

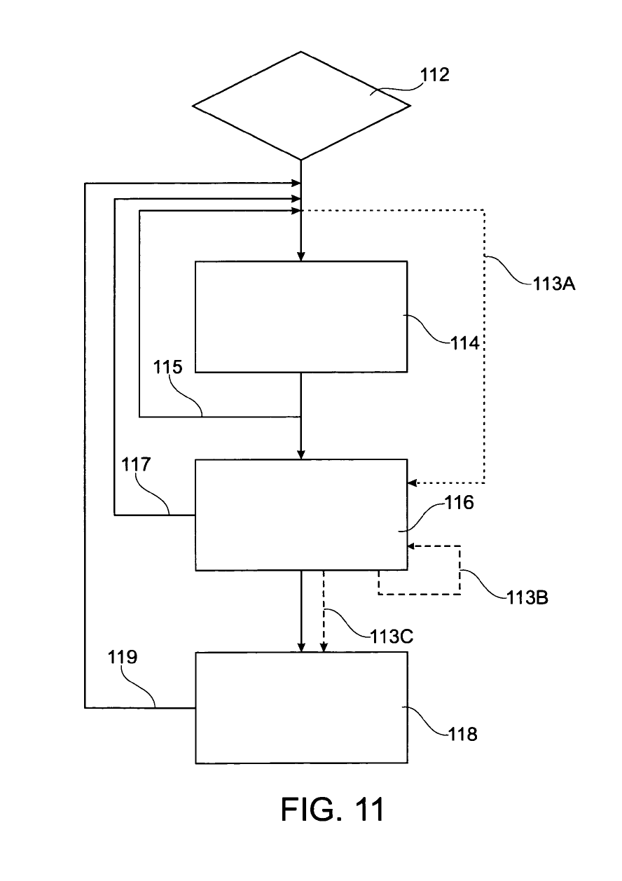

FIG. 11 shows a flow chart illustrating the benefits in treatment of atherosclerotic disease, stenotic disease, other vascular disease, e.g. stenosis or restenosis, with and according to embodiments described herein, particularly when two or more rings 820 as shown in FIGS. 7A to 10C and 12A to 13B are utilized. After diagnosing atherosclerosis (see box 112) commonly a stent implantation (see box 116) could be used as a treatment. This decision is indicated by dashed arrows 113A. Thereby, the stent pushed away the plaque in the vessel to increase the open diameter of the vessel. Unless atherosclerosis could be stopped, double stenting as indicated by arrow 113B could be used in some cases. However, typically after stenting a bypass surgery (see box 118) was typically the next treatment as indicated by arrow 113C. The stenting could prevent or postpone a bypass surgery for some time. According to some embodiments described herein, during stent implantation a medical device according to embodiments described herein can be implanted in order to monitor the status of atherosclerosis, stenotic disease, other vascular disease, e.g. stenosis or restenosis, and improve the diagnosis.

Particularly for embodiment, wherein two or more rings are used as a support structure for the sensors and thereby forming a medical device, additional benefits can be obtained. In a first treatment a balloon angioplasty (see box 114) can be provided. A balloon pushes away the plaque in a vessel. During balloon angioplasty to rings, each with at least one pressure sensor can be implanted. Thereby, atherosclerosis or other stenotic or vascular disease can be treated and at the same time a valuable monitor ability can be provided. Accordingly, a stent implantation can be postponed, whereby the risk during postponing the stent implantation can be determined by the monitoring capability of the medical devices according to some embodiments described herein. Such a surgery could be repeated, e.g. after one or two years as indicated by arrow 115.

In the event atherosclerosis or other stenotic or vascular disease could not be stopped, a stent implantation (box 116) could be postponed with a good risk management. Thereby, several years (e.g. 1 to 3) can be gained before a stent implantation and consequently also before bypass surgery (see box 118) as a further step after stent implantation. In addition for stent implantation and/or for bypass surgery, a monitoring capability can be provided by embodiments described herein. This is indicated by arrows 117 and 119. It is to be understood that the monitoring can be added in the same surgery as the stent implantation or the bypass surgery even though arrows 117 and 119 point back to the balloon angioplasty.

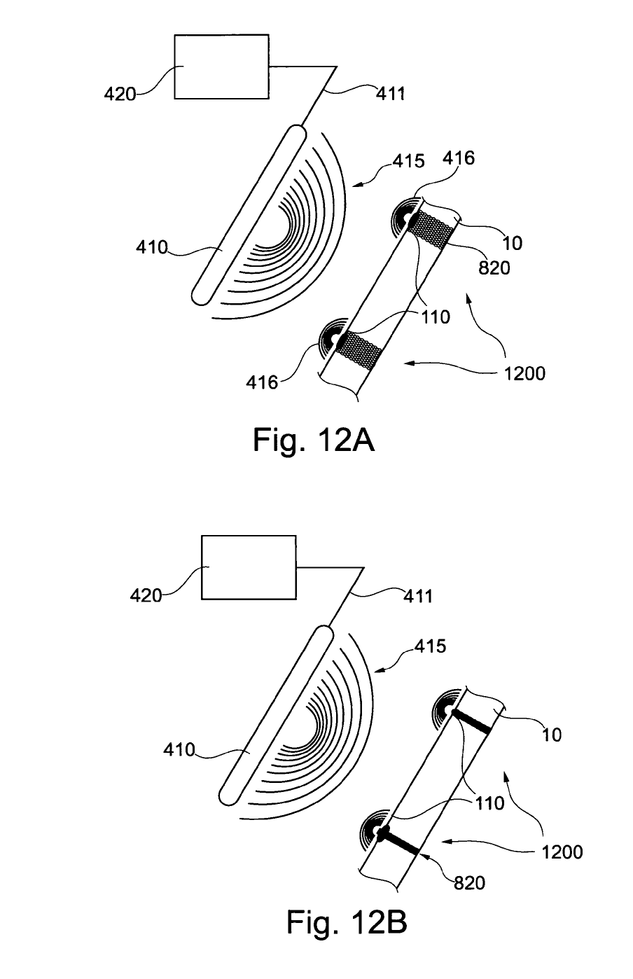

FIGS. 12A and 12B illustrate yet further embodiments described herein. A medical device for implantation 1200 includes a support structure, which can be provided by two rings 820 in the form of a short stents or two other rings. At least two sensors 110 are mounted to the support structure, wherein an axial distance is provided for the two sensors. The sensors according to embodiments described herein typically have not internal power supply. The sensors 110 are configured to sense at least the pressure inside a vascular. Optionally, one or more of a temperature, a flow rate or detection of biomarkers can be conducted as well. Thereby, for those embodiments having two or more corresponding sensors, value differences are considered at least for the pressure, the temperature and/or the flow rate.

According to typical embodiments described herein, the sensors can be provided by a MEMS (micro-electro-mechanical system). Thereby, a chip can be provided with a pressure sensor, a wireless power supply (see, e.g. RFID), a microelectronics and an antenna for transmitting the pressure values and/or further parameter values, or even a sensor ID to an external receiver or reading device. Accordingly, the sensor can include an HF frontend module for transmitting the sensed values as a function of time. The entire electronic can be provided on the chip. The sensor or chip further includes the measurement device or mechanical device, e.g. the pressure gauge, a wireless power supply, wherein the energy is transmitted by RF radiation, e.g. in the GHz range to the sensor, and optionally also a control unit and/or evaluation unit. According to alternatives a control unit and/or evaluation unit can also be provided in a remote reading device receiving the data.

As shown in FIGS. 12A and 12B, RF radiation 415 is emitted by a remote device 410. The RF radiation is used to energize the sensors 110, which in turn transmit data of measurement values and optionally other information as a wireless signal to the remote device 410 as indicated by signals 416. The remote device 410 includes the transmitter for the RF energy as well as a reading device for reading the data transmitted from the sensors 110. Alternatively, the remote RF transmitted and the reading device can also be separated. The remote device 410 is connected to a computer system 420 via connection 411. The computer system can be a personal computer, a portable computer, a server structure, a cloud structure or combinations thereof. Typically, utilizing a back-end portal structure with encrypted communication techniques allows for further data processing. Thereby, historical information and current information can be shared and compared and additional information like medication or the like can be considered in a model calculation. Those results can be shared with the user of the remote device 410 and/or a medical scientist (doctor). If critical or even emergency conditions are found, the patient, typically the user of the remote device, and/or the doctor can be informed or an alert could be generated.

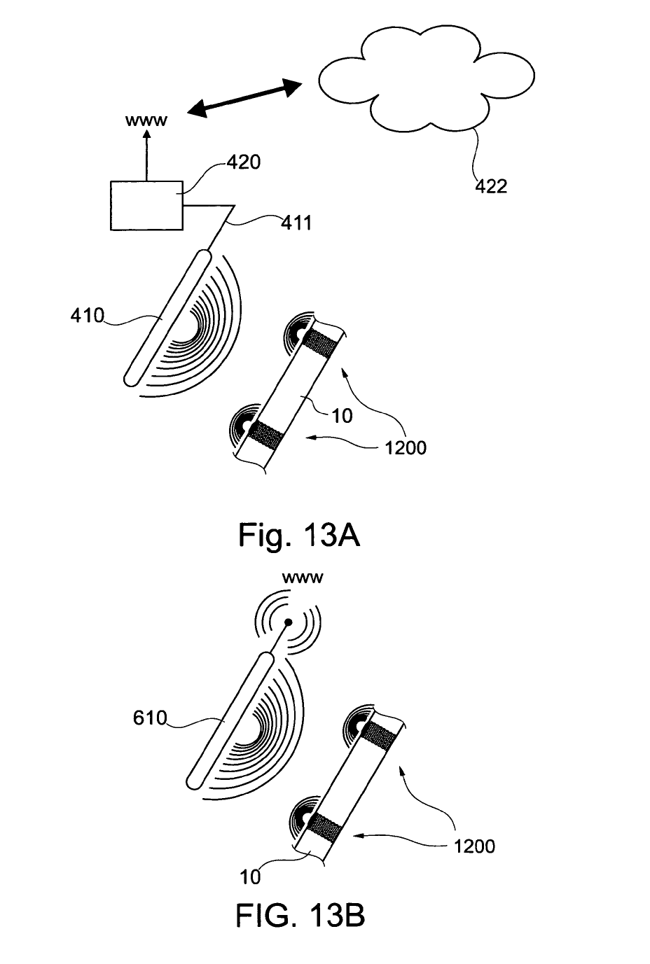

FIG. 13A shows an embodiment where a medical device 1200 having two sensor 110 is provided in a blood vessel 10. According to typical embodiments, which can be combined with other embodiments described herein, the support structure of the medical device can include two short stents or two rings. The details for power supply and data transfer from the remote device 410 to the sensors 110, which have been described above with respect to FIGS. 12A and 12B can also be incorporated in the embodiments described with respect to FIG. 13A to yield yet further embodiments. The remote device 410 is connected to a computer system 420, e.g. a laptop, which is connected to the World Wide Web and/or a server structure, e.g. in the form of a cloud solution 422. As indicated by the arrows in FIG. 13A, the server structure can communicate back to the user. The cloud solution facilitates sharing of information, e.g. with a medical expert.

FIG. 13B illustrates yet a further embodiment, where the remote device having the RF transmitter for energy transfer and the reading device for data receipt incorporated in a mobile device, such as a smart phone or the like, which can directly communicate with servers provided in a network. Modifications and implementations described above with respect to sensor arrangement, measurement methods and sensor chip design and wireless communication techniques can likewise be applied for solutions shown in FIGS. 13A and 13B.

Accordingly, embodiments described herein serve for health monitoring of atherosclerotic disease, stenotic disease, other vascular disease, e.g. stenosis or restenosis after stent implantation, stent breakage, or the like. Embodiments described herein, can yield yet further embodiments, when utilized as methods where a continuous monitoring is provided. E.g. remote device can be placed external to a human or animal body in the vicinity of or directed towards the implanted sensor(s) on a regular basis, e.g. daily or weekly or the like and the data can be transferred and evaluated accordingly. Thereby, growing restenosis can be sensed at an early stage.

The sensor or sensors provided use RF energizing techniques such as or similar to RFID chips. Thereby, the implanted sensor or sensors can be miniaturized sufficiently to also be provided in cardiovascular vessels, where stent length of 1.0 mm to 20 mm or other places at the human or animal body where stent length of 2 cm to 20 cm are utilized. According to yet further additional or alternative modifications, the implanted sensor or sensors can be miniaturized sufficiently to also be provided in vessels, where stent diameters are from 0.5 mm to 20 mm, e.g. from 1 mm to 15 mm, such as from 1 mm to 10 mm. Accordingly, the sensor can be miniaturized to a degree beyond previously known sensor for implantation. According to yet further embodiments, which can be combined with other embodiments described herein, two or more sensors are provided, wherein a pressure difference of at least two sensors is sensed. This simplifies and improves sensing of the health condition. Further, rotational positioning can be disregarded. According to yet further embodiments, which can be combined with other embodiments described herein, the support structure can be the stent or two or more stents, which are implanted for curing the vascular or cardiovascular disease. Accordingly, the health monitoring device is in place at least after the surgery for treating the original disease. Thereby, according to optional modifications, a calibration of the freshly implanted stent, e.g. an initial pressure difference can be provided.

According to yet further embodiments, which can be combined with other embodiments described herein, the sensor can be provided in a chip, e.g. as MEMS and/or as silicon based integrated HF circuits and particularly, with wireless broadband communication. Typically, the sensor can be a SoC (System on Chip), wherein sterile packaging can be provided as known in the art.

Data transmission from the sensor occurs initially to a remote device, e.g. a smart phone or another device, and is typically provided to a backend server portal, e.g. in the form of a server cloud solution. The server solution can allow, e.g. with encrypted communication such as AES, reference to a plurality of sensor information, historical data, medication information, expert information from a doctor and other information of interest for a user. The information can be graphically shown to the user for ease of understanding. Further, a cloud solution allows access from different location and also access of different persons, e.g. doctors or emergency physicians.

According to yet further embodiments, which can be combined with other embodiments described herein, a sensing system for quasi-continuous sensing as described herein and methods of monitoring, can further include a test phase with a medication model A, where a plurality of measurement cycles are conducted for a time period of days or weeks and a test phase with a medication model B, where another plurality of measurement cycles are conducted for a period of days or weeks, such that an individual medication for each patient can be developed efficiently.

While the foregoing is directed to embodiments of the invention, other and further embodiments of the invention may be devised without departing from the basic scope thereof, and the scope thereof is determined by the claims that follow.

1. A system configured for monitoring one or more health conditions, comprising:
two or more sensors configured to be implanted for exposure to blood within a vessel or to blood within a graft provided in a vessel, wherein each of the two or more sensors is configured to sense at least the pressure in the vessel or the graft;
a support structure for supporting the two or more sensors, wherein the support structure has a distal end and a proximal end, wherein at least one of the two or more sensors is closer to the distal end than another one of the two or more sensors; and
a remote device for wirelessly providing energy to the one or more sensors and for reading the pressure values.

2. The system according to claim 1, wherein at least one of the two or more sensors is mounted to the support structure adjacent to the distal end, and wherein the another one of the two or more sensors is mounted to the support structure adjacent to the proximal end.

3. A system configured for monitoring one or more health conditions, comprising:
one or more sensors configured to be implanted in a vascular, wherein the one or more sensors are configured to sense at least pressure values;
a support structure for supporting the one or more sensors;
a remote device for wirelessly providing energy to the one or more sensors and for reading the pressure values.

4. The system according to claim 1, further comprising a wireless power supply provided by one or more RFID chip, a microelectronics and an antenna for transmitting, wirelessly, the pressure values, wherein the one or more RFID chips are integrated with the sensors.

5. The system according to claim 4, wherein at least one of the sensors is mounted to the support structure adjacent to the distal end, and wherein the another one of the sensors is mounted to the support structure adjacent to the proximal end, wherein the two or more sensors and the RFID chips are employed as radiofrequency coils in magnetic resonance (MR) imaging systems, wherein the sensors and the RFID chips are configured as MRI coupling coil for intravascular imaging.

6. The system according to claim 1, wherein the sensors are provided in a chip, particularly as MEMS and/or as silicon based integrated HF circuits, and with wireless broadband communication.

7. The system according claim 4, wherein the sensors and at least one of the RFID chip and the chip are integrated with each other.

8. The system according claim 1, wherein the support structure is selected from the group consisting of: one stent, a stent arrangement comprising two or more stents, two or more rings, to or more plate patches, a graft and any of their combinations thereof.

9. The system according to claim 1, further comprising a reading device configured for wireless communication with the two or more sensors.

10. The system according to claim 3, wherein the remote device includes the reading device according to claim 9.

11. The system according to claim 9, wherein the reading device further comprises an RF emitter for providing energy to the sensors.

12. The system according to claim 9, wherein the reading device comprises a port for wired or wireless communication with a computer system.

13. The system according to claim 12, wherein the computer system is a system selected from the group consisting of: a personal computer, a portable computer, a smartphone, a server, a cloud computer system, and combinations thereof.

14. The system according to claim 9, wherein the reading device is integrated in a mobile device.

15. A method of monitoring a health condition, comprising:
wirelessly energizing at least one sensor, which senses at least vascular pressure values, with a remote device;
reading at least the pressure values for a predetermined time period via a wireless data transfer from the at least one sensor;
communicating the pressure values to a computer system;
receiving information related to the health condition from the computer system.

16. The method according to claim 15, wherein the energizing includes emitting of RF radiation.

17. The method according to claim 15, wherein the computer system is a system selected from the group consisting of: a personal computer, a portable computer, a smartphone, a server, a cloud computer system, and combinations thereof.

18. The method according to claim 15, wherein the at least one sensor is at least two sensors and the difference of respective pressure values from each of the at least two sensors is evaluated in the computer system.

19. The method according to claim 15, further comprising calibrating the at least one sensor within a second predetermined time period after a support structure implantation.

20. A system configured for monitoring one or more health conditions, particularly restenosis, comprising:
two or more sensors configured to be implanted for exposure to blood within a vessel or to blood within a graft provided in a vessel, wherein each of the two or more sensors is configured to sense at least the pressure in the vessel or the graft;
a support structure having two or more rings configured to be implanted in a vessel and for supporting the two or more sensors, wherein the support structure has a distal end and a proximal end, wherein at least one of the two or more sensors is arranged at a first ring of the two or more rings, which is closer to the distal end than a second ring of the two or more rings, wherein at least another sensor of the two or more sensors is attached to the second ring;
a remote device for wirelessly providing energy to the one or more sensors and RFID chips and for reading the pressure values; and
a reading device configured for wireless communication with the two or more sensors and RFID chips and comprising a port for wired or wireless communication with a computer system, wherein the computer system is a system selected from the group consisting of: a personal computer, a portable computer, a smartphone, a server, a cloud computer system, and combinations thereof.
